SEQUENCE LISTING (10 sequences)

SEQ ID NO: 1 - DNA, 120 bp, Homo sapiens
  CDS (1)...(120)
cagacaaaatcctgtcagtcccagccctcccaaactaaaccttctccatgcaaatctactcagcctaagccaagccagccctggcctccccagtctaagccttctcagcccagaccccct

SEQ ID NO: 2 - DNA, 3984 bp, Homo sapiens
  CDS (1)...(3984)
agtttatcagaagaacatcagcccttttctggaaaaactgcatatctgttttccccagaccactcagatgagaaactaatagaaaaagaaaatcaaattgatacagcatttttatctagcactagtaaatatgaaaagcttgaaaaacattcagcaaatcataatgttaaagatgcaactaaagaaaacagttgtgacgctaatgaagtaataaatgaaagtaattctgtatctttaagttgcataaaagaaaacataaattctagtacaggcaacgattgtgatgcaacttgcataggtcacacaaaggcgaaaactgacgtacttatatcagtcttagattcaaatgtgaagcactttttaaatgatctctaccaacaaggtaaccttattttatctgattgtaaaagaaacctggaagtaaagtggacagatcctattgagagacccatacaaaacattattacaggaaacttccttatgggcccattaaacctaactttgatagcaagtaaaaagtacagtattcctcagttatcagccgctgcagtgacagatagtgagggagaatcttcaaaatcttacttggataagcagagaattcttactgtagattcttttgcagcatccagtactgtaccacactgtgagcagagctgtagagaaaaagagcttctaaagacagaacagtgctcttcaggtaattgcctccatacagatgggaatgaaacaaatgtcactgagaattatgagttggatgtagcatcaggaactgaagaagataaaagttatggggaaaatatagtggaattatcttccagtgatagttctctgcttttaaaagataatgtaaaaggctcctcttcagaaacatgtattgtgaagaaagacactgaggacagaataacgtggaaagttaaacaagcggaaaaagcaaaagattctgtttacaaaagaagcatgactgaaggatcaactgttaatactgagtacaaaaatcaaaagaatcagatctcagaagaatcctgcttaaatgagaaaattattacaactaacttgattgattcccatctgagcactaaaaatactaccactgagtcagtccctttgaagaacacagtttctaatccgcttaacaaaagagagaagaacccccaaattaaagttagtaaagactcgcagtctgacttgacattacattcagaaatagcctatatttccaaaccaggaattctaggagttaatcatacgcctattttacctgcccactctgaaacctgtaaagtccctactcttctgaagaaacctgcgtcatacgtgagtgattttaaagaaaaacattgctcagctaatcatacggcccttatagctaatctatctcaaattttgcagagggcagatgaagcatcatctttgcagattctacaggaagaaactaaggtttgtctaaatattctccctttatttgtggaagcttttgaaagaaagcaagaatgttcagttgaacaaatcctgatttcaagagaactgttggtagaccaaaacctgtggaataattgcaaacacacattaaaaccatgtgctgttgacactttggtagaacttcaaatgatgatggaaacaattcaattcattgaaaacaaaaaaaggcacttagaaggtgaaccaacattgcgaagcttgctttggtatgatgaaacactgtatgctgagcttcttggaaaaccacgtggatttcaacagcagtctaatttctatcctggtttccaaggaagattaaaatataatgcattctgtgagttacagacttaccatgatcaattagttgaattgcttgaggaaacaaaaagggaaaagaattcatactatgtattcttaaagtacaaacgacaggttaatgaatgtgaagccataatggagcattgttccgattgctttgatttttctctttctgttccatttacctgtggagttaactttggagatagtttagaagacctggaaatcttaagaaaaagtactttaaagttgatcaatgtatgtggggactctcctaaagttcattcgtatccaggaaaacaggaccatctgtggattatcatagaaatgatctcctcaaaggttaattttattaagaacaacgaggcagtacgtgttaaaatatctctttatggtctggaacatatcttttttgatgctgcaaaaaatcttgtttggaaagagagaacacaatccttcagcaaaaaatactcacaaaagaaggacgaagaaaggctactcagagtgaataaatgtgccttttctaagttgcagaagatatatgatactttgtctaaagatttaaacaatgaaccaatttcccctattgggcttgaggaggatactataattgcttccagaaagtcagatcatccaataacgaagcacattagcatagaaaattctaaatttaacagtaatttgcttgcacacccagatatttgttgtattagtgagatattggatcaggctgaatttgcagaccttaaaaaattacaggatctcaccttgagatgtacagatcacttagaaattttaaaaaaatactttcagatgctacaagataataacatggataatatttttatcacagaagaaaatgttttagacgtggtgataaaccacagccatgaggctatcattttaaagcctgaagctattgaaatgtatattgaaatcgtcatggtctcagaaacaattcactttcttaaaaactcaatagcaaagaaactagacaaacagaggtttcgaggtatgctttggtttgatttgtcacttcttcctgagctggttcagtgccaagaaaaaatggcttctttttcatttcttaaagataactcaacagatgtttgcctttggaaagtgatagagactgctgtttccgaacttaagaaagatctggatattatctggcaaaatataatgaaagctgttaaatttgctcatatgctattcatttgcttcttcaaggagaacttcaaggaacctttcagaaataaaaaagcttctgaagaagtccaagtattaattccacatatattgacctttgtgccatatatagcatccataaattatggaagcactgtgacagagttagaatacaactacaatcaattttctacactgctgaagaatgtaatgtctgcccctaggaaagatttaggaaaaatggcccacattaggaaagtcatgaaaacgattgaacatatgaagatgatatgtactaaaaatgctgaactaaccatttcctttttcctatgccaaatgctgtataacagaaggaagattttacagctgaagagaaaagaaaaaatgaatattcatattgtaaaacctggggaaaataacaataaatttagtatttctacgatgttgcccccagtatcagagtgcataaacaaaaacatctcaaattcctctaaaaaacgaccgagcactgtagacaaatgtgaagactctcaggaacagcagcaagatactactgtttccagttgtaaaaagctaaaggtagacatgaaagatgtcacaaaaatcaacagagaaaaggcaacattcaagcatccaaggactacaggatctcatcccaaaagcgaaaacaaaatagtaccaagttcatgtgacagtctgaaaagaaatcatttaacgccaaaaaaggttgaaatgcaaagatcactacctggctcacttttacccttagagaacccaaaagacacttgcgcatcaaagtcggaaagcaaaatagacttaactgtttcatctgatcacttcagtggacaacaggaaaatttaaatagcatgaagaaaagaaatgtgaacttcagtgctgctgaaacaaaaagtgataagaaagattgtgctgcttttgcaatttgtgaccaaaaaagtgtacatggcacattttcaccagaccatgggacacttttgcagaaatttcttaaaaattcc

SEQ ID NO: 3 - DNA, 409 bp, Homo sapiens
  CDS (1)...(351)
agcttttgcagggccaaccaggctgcccgccacgtgtgcctcaaccagagcatcgacacttacaccatggtggacaaacagactctggaactctgcacatttgaagaagggagtcagatggacaacatgctggtgcaaacaaacaaaccctacaaatgcaacttgtgtgacaaaacattctccactcccaatgaggttgttaaacattcatgccaaaaccagaactcggatgtttttgccctagacgaagggcgatccattctcctgggcagtggggactcggaagtaacggagcctgaccacccagtgttagcttccatcaaaaaggaacaagaaaccgtcttactagactgaatgtcacttatctttttaaaaaactctcatttttacaaagactatcttccctccc

SEQ ID NO: 4 - DNA, 607 bp, Homo sapiens
  CDS (1)...(606)
ggaagggaaattcgtgaattgaaagaacttcaggatgaaggaagaagtgcacgattcagtgtcctcagtgtgtgtttggtaccaattgccctaaaacatttgtgcaacatgctaaaacccatgaaaaagataaaaggtactactgctgtgaagagtgaacttcaggcagtgacagaaaatgaattggaatgccatcgaggcattgcacatggggcagtggtaaaatgccctatggtcacttctgatattgcccagagaaaaacacaaaaaaagactttcatgaaagactctgtagtaggatcatccaaaaaatcagctacctacatatgtaagatgtgtccttttactacttcagccaaaagtgttttaaaaaagcacacggagtacttgcattcatcatcatgtgttgattcatttggtagtcctcttggacttgataaaagaaaaaatgacatccttgaagaacctgtagatagtgatagcactaaaacattaactaaacaacagtcaaccacatttccaaagaactctgctttaaaacaagatgtgaagcgaacatttggatcaacctcacaatcaagtagtttttcaaaaattcataagcggc

SEQ ID NO: 5 - DNA, 398 bp, Homo sapiens
  CDS (34)...(396)
tgggaccccgcgcgggctggttcgggatgagcgatggcatcggtcaaggtggccgtgagggtccggcccatgaatcgcagggaaaaggacttggaggccaagttcattattcagatggagaaaagcaaaacgacaatcacaaacttaaagataccagaaggaggcactggggactcaggaagagaacggaccaagaccttcacctatgacttttctttttattctgctgatacaaaaagcccagattacgtttcacaagaaatggttttcaaaaccctcggcacagatgtcgtgaagtctgcatttgaaggttataatgcttgtgtctttgcatatgggcaaactggatctggaaagtcatacactatgatgggaaattctggagattctggcttaat

SEQ ID NO: 6 - DNA, 2567 bp, Homo sapiens
  CDS (1)...(1122)
gtagaaactgaggaggcaggagtagtgacaacagcaacagcatctgttaatctaaaagtgagtcctaaaagaggacgacctgcagctacagaagtcaagattccaaaaccaagaggcagacccaaaatggtaaaacagccctgtccttcagagagtgacatcattactgaagaggacaaaagtaagaaaaaggggcaagaggaaaaacaacctaaaaagcagcctaagaaggatgaagagggccagaaggaagaagataagccaagaaaagagccggataaaaaagaggggaagaaagaagttgaatcaaaaaggaaaaatttagctaaaacaggggttacttcaacctccgattctgaagaagaaggagatgatcaagaaggtgaaaagaagagaaaaggtgggaggaactttcagactgctcacagaaggaatatgctgaaaggccaacatgagaaagaagcagcagatcgaaaacgcaagcaagaggaacaaatggaaactgagcagcagaataaagatgaaggaaagaagccagaagttaagaaagtggagaagaagcgagaaacatcaatggattctcgacttcaaaggatacatgctgagattaaaaattcactcaaaattgataatcttgatgtgaacagatgcattgaggccttggatgaacttgcttcacttcaggtcacaatgcaacaagctcagaaacacacagagatgattactacactgaaaaaaatacggcgattcaaagttagtcaggtaatcatggaaaagtctacaatgttgtataacaagtttaagaacatgttcttggttggtgaaggagattccgtgatcacccaagtgctgaataaatctcttgctgaacaaagacagcatgaggaagcgaataaaaccaaagatcaagggaagaaagggccaaacaaaaagctagagaaggaacaaacagggtcaaagactctaaatggaggatctgatgctcaagatggtaatcagccacaacataacggggagagcaatgaagacagcaaagacaaccatgaagccagcacgaagaaaaagccatccagtgaagagagagagactgaaatatctctgaaggattctacactagataactaggttgacatacctggaatatagagaacacttgagaagtttgtaatggttttcatttgaaatagactgctggaagtttaaatttttataagcataggtttgatgttgaaaacttgttttgagggagaaaatccctttgttttaaagtaaagtaaacattatcgctaagtgtaacttgtgcagtattaacagctacattatacagtaaatgtgggataaaatccatttagaaaatgttaaactgcttttccagacatggttgtagcatattttcaattagtgtgtgtatgttaatgtgtaattgatagtagaacaaagttacatttttaaaactgctacttgtataaaccttgcctcttttcccaaatactgtgggttttgtgcatagtttttacaaaccttggatttaccagactgtcttttcactgtttgtgggttttgtagaagttacacatttttatggtagataaaatgttacttctatacaagtactcactccctttttatcaaaagttaattttaatctcacagtctacattgtgctacattatccagcttctttggaacaatgtgtgctctgtatggttttttttggtatgacaactaattaagcaactgacattgaactgagaattctacaaactataaaacattaatttttgaaggtaatttagttttgtggctgggcattcagtgaagtcttaggacttctttgcagacaactgactgggtatatataggaatgaatctggctttagggttaaatcatttaaggtccttttataggcaggcactagtaactaaaactgaaaactaagtaagtttatttttgaggaatgttgttaaaaatgtctttaggaagtcactaaaacttaattggaagaaaaaatcatgatgcttatacaataaatatgaataaatgttatataaggaaactcacctatttgaaatcatggctatattgtttttattttctagattccaaaaatacaaacactagttgttccagcattgtactttgataagtctgtacattgacgtgtatggactaaatccagggtaaaatcaatgttacaaaatttaagggtatgttaactaaaggatagcatttctaagatattttgaatattagggtcatttggcacttctcagcaagtaggatacttctcatgtttttgaaattatatgaatatggaaaaaaatggcttaagaccagcgtctctgtatgacattgtgtggttgaccctctgagataactgttttcatctacagaattgcatttttgcttttaaagaggtcttataatggaactaggaatcaccgttttgagagaacctgcatatataccagtcattatctgtttggtccttatacagttttaacttacttagatttattctagttaagccataagttcaacgtgtaaacttgttttcattaaagaa

SEQ ID NO: 7 - DNA, 1173 bp, Homo sapiens
  CDS (1)...(543)
cccccaccatcccaacaagtcaactacattgcttcacaaccagatggaaagcaattgcagggtattcctagttcttctcatgtaagtaataacatgagtacaccagttttgcctgctccgacagcagccccaggaaatacgggaatggttcagggaccaagttctggtaatacttcgtcatcaagtcacagcaaagcctctaatgctgctgtaaaattggcagaaagcaaagtaagtgttgcagtggaagccagcgcagatagctcgaagacagacaagaaattgcaaattcaagaaaaagcagcacaagaggtaaaattggccatcaagccattttaccaaaataaagatatcaccaaggaagaatataaagaaattgtacggaaagcagtagataaagtttgtcatagtaagagtggagaagtaaattctactaaagtggcaaatctggttaaagcctatgtagacaaatacaaatattcacggaaggggagccaaaagaaaactctggaagaacctgtgtctactgaaaaaaacataggctgaaatggggaacgctgtcaaggacattatcaggatatctgcaaagtgcaatttcaacatgtaccattaactgaaaatcatacataactgtgattgaaatttggttttgataaaattatttttttaacataggatatgatgttttgttctaaataaatataggtctgcactgcaacttctgtatccttccttcccctccaccctcccccacaaaattcaagggaaagtaaagggtttaaaggaatgtgcatctttactaggactgtgttatagtgtggatactggaaaatgtatagctttttgattagggcaatggagtgcataaattagaaactttctaagtgcactggttttcaaagagatatatataatgcatttattctgtcaggttaaaatataaagtatgatctttatgattttttccctctaattatagaaagttaaataatgtattaccatgaaaaatgtttctaatattaaatagaacatatcagttgcaaagttcctaatgtgtatttttaaagcacatatctgaataaattgcctagatagaaaaaaaattatcatcgagtaaaatttagtgttcaaaacattgagacactcttcacctattgtatgaccaaataaagg

SEQ ID NO: 8 - DNA, 2131 bp, Homo sapiens
  CDS (1034)...(2131)
tgcggccgcacctgcaggcgcagagtaggtatggaagatccctcgagatccattgtgctctaaagccgccgggggtccgtgtcctgtctcggttggccggacccgggcccgagcccgagcagtagccggcgccatgtcggtggtgggcatagacctgggcttccagagctgctacgtcgctgtggcccgcgccggcggcatcgagactatcgctaatgagtatagcgaccgctgcacgccggcttgcatttcttttggtcctaagaatcgttcaattggagcagcagctaaaagccaggtaatttctaatgcaaagaacacagtccaaggatttaaaagattccatggccgagcattctctgatccatttgtggaggcagaaaaatctaaccttgcatatgatattgtgcagtggcctacaggattaacaggtataaaggtgacatatatggaggaagagcgaaattttaccactgagcaagtgactgccatgcttttgtccaaactgaaggagacagccgaaagtgttcttaagaagcctgtagttgactgtgttgtttcggttccttgtttctatactgatgcagaaagacgatcagtgatggatgcaacacagattgctggtcttaattgcttgcgattaatgaatgaaaccactgcagttgctcttgcatatggaatctataagcaggatcttcctcgcttagaagagaaaccaagaaatgtagtttttgtagacatgggccactctgcttatcaagtttctgtatgtgcatttaatagaggaaaactgaaagttctggccactgcatttgacacgacattgggaggtagaaaatttgatgaagtgttagtaaatcacttctgtgaagaatttgggaagaaatacaagctagacattaagtccaaaatccgtgcattattacgactctctcaggagtgtgagaaactcaagaaattgatgagtgcaaatgcttcagatctccctttgagcattgaatgttttatgaatgatgttgatgtatctggaactatgaatagaggcaaatttctggagatgtgcaatgatctcttagctagagtggagccaccacttcgtagtgttttggaacaaaccaagttaaagaaagaagatatttatgcagtggagatagttggtggtgctacacgaatccctgcggtaaaagagaagatcagcaaatttttcggtaaagaacttagtacaacattaaatgctgatgaagctgtcactcgaggctgtgcattgcagtgtgccatcttatcgcctgctttcaaagtcagagaattttctatcactgatgtagtaccatatccaatatctctgagatggaattctccagctgaagaagggtcaagtgactgtgaagtcttttccaaaaatcatgctgctcctttctctaaagttcttacattttatagaaaggaacctttcactcttgaggcctactacagctctcctcaggatttgccctatccagatcctgctatagctcagttttcagttcagaaagtcactcctcagtctgatggctccagttcaaaagtgaaagtcaaagttcgagtaaatgtccatggcattttcagtgtgtccagtgcatctttagtggaggttcacaagtctgaggaaaatgaggagccaatggaaacagatcagaatgcaaaggaggaagagaagatgcaagtggaccaggaggaaccacatgttgaagagcaacagcagcagacaccagcagaaaataaggcagagtctgaagaaatggagacctctcaagctggatccaaggataaaaagatggaccaaccaccccaatgccaagaaggcaaaagtgaagaccagtactgtggacctgccaatcgagaatcagctatatggcagatagacagagagatgctcaacttgtacattgaaaatgagggtaagatgatcatgcaggataaactggagaaggagcggaatgatgctaagaacgcagtggaggaatatgtgtatgaaatgagagacaagcttagtggtgaatatgagaagtttgtgagtgaagatgatcgtaacagttttactttgaaactggaagatactgaaaattggttgtatgaggatggagaagaccag

SEQ ID NO: 9 - DNA, 2106 bp, Homo sapiens
  CDS (1)...(1788)
ggatcagaaaagtttctgactctcatgaagatgccagtcatctcttttgtgcgtctaggatactgtagcttctctaagtccagaatcctcaacacacttctcagccctgcccagttgaaattacacaaaatctttcttcatcaagatttgcctcttttggtgcttccccggcaaatctctgatggcctggttgagataacatggtgttttcctgatagcgatgatagaaaggaaaacccctttttccaaaagcctgttgctctggctaatctccgtggaaatctagaaagcttttggactcagtttggttttttgatggaagtttcttcagctgtgttttttttcactgactgtttaggtgagaaggaatgggacttgctaatgtttttaggagaggctgccattgaaagatgctactttgttctcagttcccaagccagggagagtgaagaggctcaaatttttcagaggatactgaacttgaagccagcacagctactgttttgggagaggggagatgctggggatagaaggaagaacatggagggccttcaagctgccctccaggaagtgatgttctcttcttgcctcagatgtgtgtctgtggaggatatggccgccctggccagggagctggggattcaggtagatgaagactttgaaaacactcagagaattcaagtttcctctggagaaaacatggctgggacagctgaaggtgagggtcagcaaagacacagtcagctaaaaagctcatctaaaagccaggctctaatgccaattcaagagcctgggactcaatgtgagctcagccagaatcttcagaatctctatggtaccccagtattcaggcctgttctagagaactcctggctctttccaaccagaattggaggtaactttaaccatgtttccttgaaagcctcctgggttatgggccgcccctttgggtcagagcagaggcctaagtggttccatcctttgccttttcagaatgcaggggcccagggccgaggtaaaagttttggtattcaatccttccatccccagatattttattcaggtgaaagattcatgaaattttccagagttgctcggggatgtcactcgaatggaacatttgggagactgccaagacccatttgtcagcatgtacaggcctgccctgagagaccacaaatgatgggaactcttgaaaggtctagggcagtagcctccaagataggtcactcctattccctggattcacagccagcaagagcagtagggaagccatggcctcagcaagcttgcaccagggtaacagagttaactgaagcaactggaaaactgataagaacatcccatattggaaagcctcaccctcagtcctttcaaccagcagcagccacacaaaaactaagacctgcttctcagcaaggagtccagatgaagacacaaggtggggcttcaaatccagctctccaaatagggtcccatcccatgtgcaagagctctcagttcaaatccgatcagtccaacccatccacagtcaaacactcccagcctaaacccttccattctgtgccctctcaacctaaatcctctcagacaaaatcctgtcagtcccagccctcccaaactaaaccttctccatgcaaatctactcagcctaagccaagccagccctggcctccccagtctaagccttctcagcccagaccccctcaacctaagtcatcctcaaccaatccttcacaagctaaggcacaccactcaaaagcagggcagaagaggggagggaagcattaaagagctaactccagagatctataaagcatatcctttacccaggccattcctatcatatagtaagcagaagagttgccatgaaagtaaaagactactgtcattagcatgtaaaacaaagaaagatatacatgaccgaattggatatctttgtttgtttgtttgagacagagttcactcttgtgcccaggctggagtgcaatggcacgatctcggctcaccgaacctctgcttcctggcttaaagtgattctcctgctcagcctctcgagtagctgggattacaggcattgcaccaccacacccagttattttgttt

SEQ ID NO: 10 - DNA, 7450 bp, Homo sapiens
  CDS (277)...(6921)
ccccccttttttttttttaattcttcagctaaaacagcggaagaggtgatttattatatggttgttacactcggccacaaataaacacagaaatagtccagaatgtcacaggtccagggcagaggaccaacatgggcattttgtttatgagcaaggtgggtctcagaggtgatcggcgatcagagggcgatgaagttctagatccattgagacaagctctagacagtagcatgcagtcccacaacttgtaccagcatccccagcgtctggcattccatgtttctgctcctgtggcctccacggtgcaacaagctagcggtttacttggacctctgcctcatctttcttcttttgcgcttcagcctgcgcattcgcttcttcctccacttggctctcatggcgcagaggtttccagagagcctgtcacaaaagcatcctcagcactctgtggagtatgagggtaacattcatacaagtttagccattgctcaaaagctaatggaaccgaaattggggaaaataaatcaaaattatgctagcattataactgaagctttcccgaaaccaaaagacataccccaggccaaagaaatgttcattgatacagttatttcatcttataacatagaaacagctcatgacagttcaaattgcagcataactagagaacatatatgtgtccataggaaaaatgaaaatgaaccagtgtcattagagaccattcagagagactataaagaaactgcttatgttgaagataggggtcaggatcacaatctgttctgtaattcacagttaagcaatgatatatggctgaatgttaatttcaaaaaacaaacagatagagaaaaccaaaatgaggctaaagagaatagtgcttcatgtgtagaaaacaacatagagaacatatatggagacaaaaagcaggattctcatacaaacgaaaatttcagcaatatagatgaaaaggaggacaaaaattaccacaatatagaaattttgagttctgaagaattttctactaaatttaacttgatttgcagagaagataatgcagtgtcagcagcaactgcattattagagagtgaagaagataccattagtgccgtgaaacaaaaagatactgaaaatactggaagaagtgtagagcatttggcttccacgacatttcccaaaactgcaagttcttcagtgtgtgtagcctcaaatgctgcaatacagatagctagtgctactatgcctgcattaagcctaaataatgacgatcaccagatataccagtttaaagaaacttgttcttctgaaagtccagattttggtttgttagtaaaacatagggtttctgattgtgaaattgatacggataaaaataaatcacaagaatcatttcatcaatcaataaatgagaacttagttcttcagagcattgaattggaaagtgaaattgaaatagaattagaagattgtgatgatgcttttatatttcaacaagatacacatagccatgaaaacatgctttgtgaagaatttgtgacctcatataaggctctgaagtctcgtatcagttgggaaggtctgttagcacttgataacggggagatggaagttttggaaagcaccacaggaagggagaatagtgatcagcattattctaaggaaagtaactatttttattcctctacacaaaacaatgaaacagaacttaccagcccaattttacttccagatctacaaattaaaattactaatatatttaggccaggattcagcccgacagctgactcccttgcattgaaagatagtttttgcacacatgtaactgaagccacaaaaccggaaataaataaggaagatggagaaattctaggatttgacatttattcccagccttttggtgaaaatgcagattatccatgtgaagataaagttgataatataaggcaagaatcagggccagtgagtaactctgaaatctccctttcttttgacttgagtcgtaatacagatgtgaatcatacgtctgaaaatcagaacagtgaatctttgtttactgaaccttctaatgtcacaacaatagatgatggaagcagatgtttctttacaaaatcaaaaactgactataatgataccaaaaataaaaaggaggtagaatcaagaattagcaaaaggaagctacatatatcttccagggatcagaacataccacataaagatttaagacgacataaaatttatgggagaaagaggaggctaaccagtcaagactcatctgagtgtttctcttcattatcccaaggacgaattaaaacattttcacagtcagaaaagcacattaagagtgtcctaaatatcctaagtgatgaagcatctttatgtaaaagcaaatgtctttccagaaaactagacaaagcagttgttcacttaaaaaaagctcatagaagagttcacacatctttgcagcttataactaaagtaggagaagaaagaaagggcccattaccaaaatcatatgcaataatatgcaataatttctgggaaagttgtgaccttcaaggttatagttctgtgtctcaaagaaaatattattctactaagcatttttcgtcaaaaagaaaatatgacaaacggagaaagaaaagagctccaaaagctgatatttctaaatcattaacccatgtgtcaaagcacaagtcttataaaacaagtggagagaaaaaatgcctttctaggaaaagtatggctagcagtgtctcaaaaagtcaccccaccaccagtcacatgggagaattttgtaatcaagaacatcctgaatcacagttgcctgtatcctccacatcccaaagtacaagtcagtcagtttattataatagcagtgtaagcaatccaagtttatcagaagaacatcagcccttttctggaaaaactgcatatctgttttccccagaccactcagatgagaaactaatagaaaaagaaaatcaaattgatacagcatttttatctagcactagtaaatatgaaaagcttgaaaaacattcagcaaatcataatgttaaagatgcaactaaagaaaacagttgtgacgctaatgaagtaataaatgaaagtaattctgtatctttaagttgcataaaagaaaacataaattctagtacaggcaacgattgtgatgcaacttgcataggtcacacaaaggcgaaaactgacgtacttatatcagtcttagattcaaatgtgaagcactttttaaatgatctctaccaacaaggtaaccttattttatctgattgtaaaagaaacctggaagtaaagtggacagatcctattgagagacccatacaaaacattattacaggaaacttccttatgggcccattaaacctaactttgatagcaagtaaaaagtacagtattcctcagttatcagccgctgcagtgacagatagtgagggagaatcttcaaaatcttacttggataagcagagaattcttactgtagattcttttgcagcatccagtactgtaccacactgtgagcagagctgtagagaaaaagagcttctaaagacagaacagtgctcttcaggtaattgcctccatacagatgggaatgaaacaaatgtcactgagaattatgagttggatgtagcatcaggaactgaagaagataaaagttatggggaaaatatagtggaattatcttccagtgatagttctctgcttttaaaagataatgtaaaaggctcctcttcagaaacatgtattgtgaagaaagacactgaggacagaataacgtggaaagttaaacaagcggaaaaagcaaaagattctgtttacaaaagaagcatgactgaaggatcaactgttaatactgagtacaaaaatcaaaagaatcagatctcagaagaatcctgcttaaatgagaaaattattacaactaacttgattgattcccatctgagcactaaaaatactaccactgagtcagtccctttgaagaacacagtttctaatccgcttaacaaaagagagaagaacccccaaattaaagttagtaaagactcgcagtctgacttgacattacattcagaaatagcctatatttccaaaccaggaattctaggagttaatcatacgcctattttacctgcccactctgaaacctgtaaagtccctactcttctgaagaaacctgcgtcatacgtgagtgattttaaagaaaaacattgctcagctaatcatacggcccttatagctaatctatctcaaattttgcagagggcagatgaagcatcatctttgcagattctacaggaagaaactaaggtttgtctaaatattctccctttatttgtggaagcttttgaaagaaagcaagaatgttcagttgaacaaatcctgatttcaagagaactgttggtagaccaaaacctgtggaataattgcaaacacacattaaaaccatgtgctgttgacactttggtagaacttcaaatgatgatggaaacaattcaattcattgaaaacaaaaaaaggcacttagaaggtgaaccaacattgcgaagcttgctttggtatgatgaaacactgtatgctgagcttcttggaaaaccacgtggatttcaacagcagtctaatttctatcctggtttccaaggaagattaaaatataatgcattctgtgagttacagacttaccatgatcaattagttgaattgcttgaggaaacaaaaagggaaaagaattcatactatgtattcttaaagtacaaacgacaggttaatgaatgtgaagccataatggagcattgttccgattgctttgatttttctctttctgttccatttacctgtggagttaactttggagatagtttagaagacctggaaatcttaagaaaaagtactttaaagttgatcaatgtatgtggggactctcctaaagttcattcgtatccaggaaaacaggaccatctgtggattatcatagaaatgatctcctcaaaggttaattttattaagaacaacgaggcagtacgtgttaaaatatctctttatggtctggaacatatcttttttgatgctgcaaaaaatcttgtttggaaagagagaacacaatccttcagcaaaaaatactcacaaaagaaggacgaagaaaggctactcagagtgaataaatgtgccttttctaagttgcagaagatatatgatactttgtctaaagatttaaacaatgaaccaatttcccctattgggcttgaggaggatactataattgcttccagaaagtcagatcatccaataacgaagcacattagcatagaaaattctaaatttaacagtaatttgcttgcacacccagatatttgttgtattagtgagatattggatcaggctgaatttgcagaccttaaaaaattacaggatctcaccttgagatgtacagatcacttagaaattttaaaaaaatactttcagatgctacaagataataacatggataatatttttatcacagaagaaaatgttttagacgtggtgataaaccacagccatgaggctatcattttaaagcctgaagctattgaaatgtatattgaaatcgtcatggtctcagaaacaattcactttcttaaaaactcaatagcaaagaaactagacaaacagaggtttcgaggtatgctttggtttgatttgtcacttcttcctgagctggttcagtgccaagaaaaaatggcttctttttcatttcttaaagataactcaacagatgtttgcctttggaaagtgatagagactgctgtttccgaacttaagaaagatctggatattatctggcaaaatataatgaaagctgttaaatttgctcatatgctattcatttgcttcttcaaggagaacttcaaggaacctttcagaaataaaaaagcttctgaagaagtccaagtattaattccacatatattgacctttgtgccatatatagcatccataaattatggaagcactgtgacagagttagaatacaactacaatcaattttctacactgctgaagaatgtaatgtctgcccctaggaaagatttaggaaaaatggcccacattaggaaagtcatgaaaacgattgaacatatgaagatgatatgtactaaaaatgctgaactaaccatttcctttttcctatgccaaatgctgtataacagaaggaagattttacagctgaagagaaaagaaaaaatgaatattcatattgtaaaacctggggaaaataacaataaatttagtatttctacgatgttgcccccagtatcagagtgcataaacaaaaacatctcaaattcctctaaaaaacgaccgagcactgtagacaaatgtgaagactctcaggaacagcagcaagatactactgtttccagttgtaaaaagctaaaggtagacatgaaagatgtcacaaaaatcaacagagaaaaggcaacattcaagcatccaaggactacaggatctcatcccaaaagcgaaaacaaaatagtaccaagttcatgtgacagtctgaaaagaaatcatttaacgccaaaaaaggttgaaatgcaaagatcactacctggctcacttttacccttagagaacccaaaagacacttgcgcatcaaagtcggaaagcaaaatagacttaactgtttcatctgatcacttcagtggacaacaggaaaatttaaatagcatgaagaaaagaaatgtgaacttcagtgctgctgaaacaaaaagtgataagaaagattgtgctgcttttgcaatttgtgaccaaaaaagtgtacatggcacattttcaccagaccatgggacacttttgcagaaatttcttaaaaattcccacagggttgtttataatagcaatatacatacacatttggctagtactaggtgaataggaaaataaatcatgctgtatgtatacaataagaggtcaagttgccaataaattattactgttaatgttctgggaaatgctgaaactatgctaagtgggggagagggaaagcaggtattgcagttttgtagtgaagatgggctttggagtcatatctgagatgtaagtagcagcttttaaattctagctatgaccctgtgcagatcacttaacttttgagtgtcaggatgtttggaaggacaagacaggaaagtgttttaatacagggttcagtatttagtaagccttcaataagtgatattacatacatgtaataaaaactaagcattgaaaggactaggaaaaatgcatcaaatgttagttctgcaggttatagaattatacttttatgtaattttaaaatttcagttatgaacatgtttgtccattacaaacttataatagctgaaaaacagggttgtgagaacagcttcagttaaaattgtcctgtctagttttgaatattcaagaacagaaagccttt

(19) United States
(12) Patent Application Publication
(10) Pub. No.: US 2003/0008330 A1
(43) Pub. Date: Jan. 9, 2003

(54) Polynucleotides, polypeptides, remedies for cancer and vaccines

(76) Inventors: Akihiro Abe, Nagoya-shi (JP); Nobuhiko Emi, Nagoya-shi (JP); Hidehiko Saito, Nagoya-shi (JP)

Correspondence Address: Y Rocky Tsao, Fish & Richardson, 225 Franklin Street, Boston, MA, 02110-2804, US

(21) Appl. No.: 10/149,110

(22) PCT Filed: Dec. 13, 2000
(86) PCT No.: PCT/JP00/08813

(30) Foreign Application Priority Data
Dec. 13, 1999 (JP) 11/353688

(51) Int. Cl. G01N 33/567; C12Q 1/68; C07H 21/04; C12N 9/00; C12N 5/06; C07K 16/40

(52) U.S. Cl. 435/7.21; 435/6; 435/326; 435/183; 530/388.26; 536/23.2

(57) ABSTRACT

The present invention provides polynucleotides, polypeptides, remedies for cancer, and vaccines, which are used for diagnosing autoimmune diseases such as graft-versus-host disease, and graft-versus-leukemia, and treating and preventing acute or chronic myeloid leukemia. The polynucleotides includes a base sequence that is identical or substantially identical with a base sequence represented by SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10. The polypeptide includes an amino acid sequence that is identical or substantially identical with an amino acid sequence encoded by the above-mentioned polynucleotide. The remedy for cancer contains the polynucleotides or polypeptides, and strengthens the defense mechanism in a living body to thereby treat cancer. The vaccine contains the polynucleotides or polypeptides, and makes living bodies to acquire the defense mechanism to thereby prevent the onset of cancer.

### TECHNICAL FIELD

The present invention relates to polynucleotides, polypeptides, remedies for cancer, and vaccines used for diagnosis of autoimmune diseases such as graft versus host disease (hereafter referred as “GVHD”) and graft versus leukemia (hereafter referred as “GVL”), and for treatment and prevention of cancers such as acute myelogenous leukemia (hereafter referred as “AML”) and chronic myelogenous leukemia (hereafter referred as “CML”).

### BACKGROUND ART

GVHD, which is a kind of autoimmune disease characterized by graft versus host reaction, is a disease in which lymphatic system immunocytes (especially, T cells) of a transplanted donor immunologically attack tissue cells of a recipient (a host) whose gene organization differs, and the tissues of the recipient receive damages. The GVHD frequently occurs after bone marrow transplantation (BMT) for an AML or a CML patient due to the difference of human leukocyte antigen (HLA) between the donor and the recipient. On the other hand, GVL is a kind of autoimmune disease, and is caused by lymphatic system immunocytes of the donor immunologically attacking leukemia cells in the recipient after bone marrow transplantation for the AML or the CML patient.

Recently, an approach in which cDNA (complementary DNA) library of a human cell or a human cell line is screened using serum of these autoimmune disease patients and then oncogene and target gene of autoimmune disease are identified, has been noticed, and many genes have ever been identified. A polypeptide, which is a product of these genes, includes an antigen that is at least recognized by helper T cells in a living body of the patient. Therefore, the polypeptide has high possibility of becoming a key that causes cancers such as AML and CML and autoimmune diseases such as GVHD and GVL.

### DISCLOSURE OF THE INVENTION

The objectives of the present invention are to provide polynucleotides, polypeptides, remedies for cancer and vaccines used for diagnosis of autoimmune diseases such as GVHD and GVL and for treatment and prevention of AML or CML.

To achieve the above objectives, the first aspect of the present invention includes a polynucleotide comprising a base sequence that is identical or substantially identical with a base sequence represented by SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

Another aspect of the present invention includes a recombinant vector containing the above-mentioned polynucleotide.

The further aspect of the present invention includes a transformant containing the above-mentioned recombinant vector.

The polynucleotide of the present invention may be used with labeled with radioisotope, enzyme, or fluorochrome.

The further aspect of the present invention includes a polypeptide comprising an amino acid sequence that is identical or substantially identical with an amino acid sequence encoded by the above-mentioned polynucleotide.

The further aspect of the present invention includes an antibody against the polypeptide of the present invention.

The further aspect of the present invention includes a hybridoma producing an antibody of the present invention.

The further aspect of the present invention includes a diagnostic product for autoimmune diseases containing a polypeptide of the present invention.

The further aspect of the present invention includes a diagnostic kit of autoimmune diseases, which contains the polypeptide of the present invention, for screening serum of a GVHD or a GVL patient.

The further aspect of the present invention includes a kit for examining diseases, which contains the antibody of the present invention, for analyzing living tissues of an AML, a CML, a GVHD, or a GVL patient.

The further aspect of the present invention includes a remedy for cancer, which contains the polynucleotide of the present invention or the polypeptide of the present invention, for treating a cancer by strengthening the defense mechanism of a living body against cancer cells.

The further aspect of the present invention includes a vaccine, which contains the polynucleotide of the present invention or the polypeptide of the present invention, for preventing a cancer by allowing a living body to acquire the defense mechanism of the living body against cancer cells.

The further aspect of the present invention includes a DNA chip containing the above-mentioned polynucleotide.

### BEST MODE FOR CARRYING OUT THE INVENTION

The present embodiments will be described below in detail.

The polynucleotide of the present embodiment includes a base sequence represented by SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO:. 8, SEQ ID NO: 9, and SEQ ID NO: 10. The polynucleotide represented by SEQ ID NO: 1 to SEQ ID NO: 10 is a part of human genome cloned from human leukemia K562 cells.

The polynucleotide including a base sequence represented by SEQ ID NO: 1 and SEQ ID NO: 2 (hereafter referred as “pn1” and “pn2”) encodes a gene product specifically causing antigen-antibody reaction with an antibody contained in serum of an AML patient who developed GVHD after the bone marrow transplantation and is still in a condition of developing GVHD. The polynucleotide including a base sequence represented by each of SEQ ID NO: 3 to SEQ ID NO: 8 (hereafter referred as “pn3” to “pn8”) encodes a gene product specifically causing antigen-antibody reaction with an antibody contained in serum of a CML patient.

The polynucleotide including a base sequence represented by SEQ ID NO: 9 (hereafter referred as “pn9”) encodes a continuous base sequence of a genome of K562 cells including a base sequence represented by SEQ ID NO: 1. 1-1788 in the sequence is likely to encode open reading frame (ORF). Here, the pn9 is predicted to be confirmed an initiation codon at a position where it slightly extends to an N-terminal side. The polynucleotide including a base sequence represented by SEQ ID NO: 10 (hereafter referred as “pn10”) encodes a continuous base sequence of a genome of K562 cells including a base sequence represented by SEQ ID NO: 2. 277-6921 in the sequence is likely to encode open reading frame (ORF).

The polynucleotide of the present embodiment includes a base sequence that is substantially identical with a base sequence represented by SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

“a base sequence that is substantially identical with” is an encoded base sequence similar to a base sequence represented by the SEQ ID NOs. having at least one of slight differences selected from deletion, addition, and substitution of partial bases, and has preferably more than 80% homology, more preferably more than 90% homology, and even more preferably more than 95% homology. Alternatively, it contains preferably more than 50%, more preferably more than 75%, and even more preferably more than 95% of base sequences with preferably more than 80% homology, more preferably more than 90% homology, and even more preferably more than 95% homology with respect to the overall length of a base sequence represented by each of the SEQ ID Nos.

A polynucleotide of the present embodiment may be a polynucleotide fragment or an oligonucleotide fragment obtained by cutting the above-mentioned polynucleotide using various restriction enzymes such as endonuclease, exonuclease, and the like. The polynucleotide fragment or the oligonucleotide fragment may preferably have a length of more than 20 bases, more preferably more than 50 bases, and even more preferably more than 100 bases, and especially preferably more than 500 bases.

In addition, the substantially identical base sequence may preferably encode an identical or a substantially identical amino acid sequence. “an amino acid sequence that is substantially identical with” is an encoded amino acid sequence having at least one of slight differences selected from deletion, addition, and substitution of partial amino acids, and has preferably more than 80% homology, more preferably more than 90% homology, even more preferably more than 95% homology, and most preferably more than 98% homology.

The polynucleotide may be stored and amplified in the condition that it is inserted in the predetermined position of a widely used vector in the form of double strand DNA. As the above-mentioned vectors, for example, plasmid vectors derived from Escherichia coli, yeast, or Bacillus subtilis, bacteriophage vectors such as phage λ, or virus vectors such as a retrovirus, an adenovirus, a vaccinia virus, and a baculovirus may preferably be used.

When the polynucleotide is amplified, a vector including such polynucleotide is initially constructed. Next, the vector is incorporated into an appropriate host cell to produce a transformant, and the transformant is proliferated to amplify the polynucleotide. The above-mentioned host cell may include, for example, bacteria (genus Escherichia, genus Bacillus), yeasts, insect cells, animal cells, or mammalian cells. The vectors and the polynucleotides may easily and massively be produced using these transformants, and then gene product of the polynucleotide may also easily and massively be produced.

Alternatively, the polynucleotide may massively be obtained by extracting total RNA or messenger RNA (mRNA) fraction from K562 cells, other human cell line, human culture cells, or human tissues, and directly amplifying the extracted solution using RT-PCR method(Reverse Transcriptase Polymerase Chain Reaction Method). Alternatively, the polynucleotide may be obtained by artificial synthesis using an automated DNA synthesis machine.

On the other hand, the polynucleotide may be used as an antisense DNA or an antisense RNA that is a complementary sequence with respect to a base sequence that is normally expressed as mRNA. More specifically, when at least part of the base sequence of the polynucleotide has a sequence specific for cancer cells of an AML or a CML patient, the specific sequence or a neighboring sequence of the specific sequence including the specific sequence may be used for diagnosis of AML or CML, and thus may assist subsequent treatment. That is, the above-mentioned sequence may be used for a diagnostic probe for well-known methods such as Southern hybridization method, Northern hybridization method, and PCR (Polymerase Chain Reaction) method, after the cancer cells are obtained from the AML or the CML patient. Tissues obtained from the above-mentioned patient may also be used for analyzing expression site or expression condition of the polynucleotide including the above-mentioned sequence, and localization on the human chromosome using in situ hybridization method. Here, the polynucleotide may normally used with labeled with radioisotope, enzyme, or fluorochrome.

The antisense DNA and the antisense RNA may be used as a remedy for alleviating symptom of autoimmune diseases such as GVHD, GVL, and the like by suppressing the expression level of mRNA of the polynucleotides recognized as an antigen so that the amount of the resultant gene product decreases. When a polynucleotide is one that encodes a polypeptide relating to crisis of a cancer, it may be used as a remedy for cancer for suppressing proliferation of cancer cells by suppressing the production amount of the gene product of the polynucleotide.

The antisense DNA or the antisense RNA may preferably be administered into a human body independently or with a form in which it is inserted into a retrovirus vector, an adenovirus vector, or an adeno-associated virus vector.

The polypeptide of the present embodiment includes an amino acid that is identical or a substantially identical with an amino acid encoded by the above-mentioned polynucleotide. That is, it includes an amino acid that is identical or a substantially identical with an amino acid represented by SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

“an amino acid sequence that is substantially identical with” is an encoded amino acid sequence similar to an amino acid sequence having at least one of slight differences selected from deletion, addition, and substitution of partial amino acids. It is encoded with an amino acid sequence having preferably more than 80% homology, more preferably more than 90% homology, even more preferably more than 95% homology, and most preferably more than 98% homology.

More specifically, when an amino acid sequence is an encoded one having slight differences through the substitution, the property of the amino acid to be substituted may preferably be similar to that of a substituted amino acid. As an example of such pairs of an amino acid to be substituted and a substituted amino acid, glycine and alanine; valine, leucine, and isoleucine; proline, phenylalanine, and tryptophan; aspartic acid and glutamic acid; asparagine and glutamine; serine, threonine, and tyrosine; or lysine, arginine, and histidine may be included. In these substitutions, tertiary structure of a polypeptide is similar in many cases. Therefore, there is high possibility of being recognized to an identical antibody, and there is high possibility of working in a living body of a patient as an antigen that causes autoimmune disease such as GVHD and GVL.

The polypeptide of the present embodiment may be a polypeptide fragment or an oligopeptide fragment obtained by cutting the polypeptide using a proteolytic enzyme such as protease, proteinase, endopeptidase, exopeptidase, and the like. These polypeptide fragments or oligopeptide fragments have high possibility of working as an antigen that causes autoimmune diseases in a living body of a patient.

In general, the polypeptides may easily and massively be obtained by compulsorily expressing the polynucleotide inserted into the transformant. Alternatively, it may be obtained by artificially synthesizing with solid phase synthesis method or liquid phase synthesis method. Alternatively, it may also be obtained by fractioning and purifying protein extraction fraction extracted from K562 cells, other human cell line, human culture cells, or human tissues using affinity chromatography carrier with which an antibody against the polypeptide, which is produced in advance, is combined.

The antibody may include a monoclonal antibody or a polyclonal antibody. The polyclonal antibody may be preferable for increasing diagnosis sensitivity, while the monoclonal antibody may be preferable for detailed diagnosis or analysis. The polyclonal antibody is generally obtained by multiply immunizing an immune animal such as a goat, a rabbit, cattle, and a horse with the polypeptide together with an adjuvant if necessary, and collecting serum from the immune animal. The monoclonal antibody is generally obtained by multiply immunizing an immune animal such as a mouse, a rat, a goat, and a rabbit with the polypeptide together with an adjuvant if necessary, and collecting B cells from the immune animal, cell-fusing the B cells with myeloma cells to produce hybridoma, and obtaining culture supernatant of a single hybridoma that was cloned.

These antibodies (primary antibodies) may be used as a diagnostic tool or an analyzing tool for analyzing localization where the polypeptide is expressed in tissues or cells (i.e., immunohistochemical staining) with secondary antibodies that are labeled with radioisotope, enzyme, fluorochrome, or the like. Also, it may be used for identification of an antigen that causes GVHD or GVL, or quantitation of the expression level of the polypeptide. When the polypeptide includes physiological activity such as enzymes and the antibody is a neutralizing antibody for such polypeptide, the antibody may be used, for example, as a remedy for cancer since it inhibits the physiological activity of the polypeptide that works as an antigen.

The remedy for cancer of the present embodiment contains the above-mentioned polynucleotide or polypeptide, and immunologically treats the cancer by strengthening the defense mechanism of a living body against cancer cells in the living body of a cancer patient. The defense mechanism of the living body includes at least one of immune systems selected from antibody-mediated immunity targeting a gene product from the polynucleotide or the polypeptide, and cell-mediated immunity. The cancer cell exposes on the surface of cells in a form in which it recognizes the target as an immunologically not-self, and the main target of the remedy for cancer includes cancer cells of an AML or a CML patient.

The vaccine of the present embodiment contains the above-mentioned polynucleotide or polypeptide, and immunologically prevents a cancer by allowing a living body to acquire the defense mechanism of the living body against cancer cells. The defense mechanism of the living body includes an identical immune system with that for the remedy for cancer. The vaccine may be used for acquiring the preliminary defense mechanism of a living body to inhibit the occurrence of cancer cells that are exposed on the surface of a cell in the form in which the vaccine recognizes the target as an immunologically not-self. It may mainly be used for preventing morbidity for AML or CML.

The remedy for cancer or the vaccine of the present embodiment may be used as a mixed remedy for cancer or a mixed vaccine by appropriately mixing at least one selected from the above-mentioned polynucleotides with at least one selected from the above-mentioned polypeptides. In addition, it is preferable that the mixed remedy for cancer may simultaneously contain a polypeptide as a tumor antigen that is included in cancer cells, which is a target for the treatment, and a polynucleotide that encodes the polypeptide.

The remedy for cancer or the vaccine may be used, for example, in the dosage form of an injection. The injection may contain at least one selected from the polynucleotide and the polypeptide in an isotonic solution containing physiological saline, grape sugar and an adjuvant (e.g., sorbitol and mannitol). If necessary, the injection may include at least one selected from solubilizing agent, buffer for holding pH of a solution constantly, stabilizer (antioxidant) and preservative for improving the stability, and an analgesic agent for alleviating pain during the injection.

As the solubilizing agent, for example, alcohols such as ethanol, polyethylene glycol, and propylene glycol; nonionic surfactants; fats and oils such as sesame oil and soybean oil; benzyl benzoate; and benzyl alcohol may preferably be used. Preferably, the stabilizer, the preservative and the analgesic agent listed in Japanese pharmacopoeia may be used. In addition, at least one selected from various vitamins, well-known pharmaceutical formulations used for the cancer treatment such as interferons α to γ, physiologically active substances for activating the immune system such as interleukin 1 (α, β), interleukins 2 to 8 and other cytokines may preferably be contained.

The operations of the above embodiments will now be described.

When a patient of autoimmune disease such as GVHD or GVL or a person who is suspicious for the autoimmune disease (hereafter, both referred as “a person to be diagnosed”) is diagnosed using the polypeptide, blood from the person to be diagnosed is initially obtained to fractionate its serum. Next, the diagnosis of the autoimmune disease is carried out through examining the antigen-antibody reaction between antibody contained in the serum and the polypeptide.

Here, for example, quantitative reactions such as immunoprecipitation reaction, double gel diffusion method, and immunoelectrophoresis; and quanitative reactions such as quantitative precipitation reaction, radioimmunoassay, and enzyme-linked immunosorbent assay may be used as the antigen-antibody reaction. When the polypeptide or the protein including the polypeptide has physiological activity such as enzyme, it is possible to confirm the specific antigen-antibody reaction by examining the reduction of the physiological activity and also to prove that the antigen contained in the serum is a neutralizing antibody. In addition, after confirming the existence of the antibody against the polypeptide in the serum of the person to be diagnosed, the tissue is obtained from the person to be diagnosed and the localization of the antigen may immunohistologically be analyzed using an immunohistochemical staining method.

On the other hand, when the injection containing the polynucleotide as a remedy for cancer is administered into a living body of the AML or the CML patient, the polynucleotide is incorporated into the cells of the patient, is translated, and a resultant gene product is produced. At least part of the produced gene product is processed within cells in various manners and then is fragmented small. Subsequently, the fragmented product is combined with a class I or a class II HLA protein that exists in the cell membrane surface, and then is recognized by T cells as a tumor antigen.

When the fragmented gene product is combined with the class I HLA protein, cytotoxic T lymphocytes (CTL) in the cancer patient's body recognize the fragment with the tumor antigen, and kill the cells. In addition, this stimulates the CTL, and the CTL is rapidly cell-divided to proliferate. Subsequently, the increased number of CTL after the proliferation circulates in a living body of the patient, finds cells presenting the similar fragment on the HLA protein, i.e., cancer cells, and then kills these cells.

When the fragmented gene product is combined with the class II HLA protein, helper T cells existing in the patient's body recognize the fragment with a tumor antigen. The information that the fragment is the tumor antigen stimulates B cells to produce antibodies, and transmits the proliferative stimulus to the CTL. Subsequently, the B cells are rapidly cell-divided to proliferate, and then the antibodies that are more specifically combined with the fragment are massively produced. Then, these antibodies are specifically combined with the fragment or the gene product of the polynucleotide, and then immunological attack against the fragment and the gene product by phagocytes such as macrophages and complements of the patient's body may be promoted. When the gene product has physiological activity, the combination of the antibody neutralizes its physiological activity.

When the injection containing the polypeptide is administered into a living body of the AML or the CML patient as a remedy for cancer, most of the polypeptides are recognized by helper T cells and B cells as a tumor antigen, and a part of the polypeptides are incorporated into cells (mainly, the phagocytes) of the patient's body, and are presented on the cell surface together with class I or class II HLA protein. As a result, as described above, cancer cells are dead through the attack by the immune system including the CTLs, the B cells, the phagocytes, and complements.

In addition, when the mixed remedy for cancer is administered into a living body of the AML or the CML patient, cell group of immune system is activated against both the gene product of the polynucleotide and the polypeptide, and then additive effect or synergistic effect is displayed. On the other hand, a part of cell group of the immune system (helper T cells, CTLs, B cells) activated by the administration of the remedy for cancer keeps circulating in the living body of the cancer patient as a memory cell, and prevent survived cancer cells or cancer cells that present a similar fragment from proliferating or developing again.

When a vaccine containing the polynucleotide (a DNA vaccine) is administered into a living body of a human in the form of an injection, the polynucleotide is incorporated in cells of the human's body, and then is translated. As a result, a gene product of the polynucleotide is expressed. As described above, T cells recognize the resultant gene product as an antigen together with class I or class II HLA protein. Therefore, the process of a series of weak immune system activation is passed to exclude the antigen, and finally memory cells are produced. When the vaccine containing the polypeptide is administered into the living body of the human in the form of an injection, the process of a series of weak immune system activation is also passed, and finally memory cells are produced.

In addition, when the mixed vaccine is administered into a living body of the AML or the CML patient, cell group of immune system is activated against both the gene product of the polynucleotide and the polypeptide, and then additive effect or synergistic effect is displayed. It is preferable that these vaccines are multiply administered at a predetermined interval. As a result, the specificity of the memory cells with respect to the fragment or the gene product is further increased, and the c of cancer cells presenting the similar fragment is further strongly inhibited.

The above described embodiments have the following effects.

The polynucleotide of the present invention includes a base sequence that is identical or substantially identical with a base sequence represented by SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10. It is possible to treat a cancer by administering these polynucleotides to an AML or a CML patient, and then strengthening the defense mechanism of a living body of the patient against the cancer cells. In addition, it is possible to prevent development and proliferation of the cancer cells by administering these polynucleotides to a person other than the patient to have the person acquire defense mechanism of a living body against the cancer cells that causes AML or CML.

The recombinant vector of the present invention is a recombinant vector containing a polynucleotide of the present invention. The recombinant vector allows the polynucleotide to be preserved easily and stably.

The transformant of the present invention is a transformant containing the recombinant vector of the present invention. The transformant allows the polynucleotide and the vector to be easily amplified to obtain them.

The polynucleotide of the present invention may be labeled with radioisotope, enzyme or fluorochrome. It is possible to analyze expression of the polynucleotide in the living tissues with high sensitivity, and to assist diagnosis and treatment.

The polypeptide of the present invention includes an amino acid sequence that is identical or substantially identical with an amino acid sequence encoded by the above-mentioned polynucleotide. It is possible to treat a cancer by administering these polypeptides to an AML or a CML patient, and then strengthening the defense mechanism of a living body of the patient against the cancer cells. In addition, it is possible to prevent development and proliferation of the cancer cells by administering these polynucleotides to a person other than the patient to have the person acquire defense mechanism of a living body against the cancer cells that causes AML or CML. Further, the diagnosis of GVHD or GVL may easily be carried out by examining whether the antigen-antibody reaction occurs against serum of a person to be diagnosed using these polypeptides.

The antibody of the present invention is an antibody against the polypeptide of the present invention. The use of the antibody may easily analyze the localization of the polypeptide in the living tissues, and thus may assist diagnosis and treatment.

The hybridoma of the present invention is a hybridoma producing an antibody of the present invention. Monoclonal antibodies, which are able to analyze the localization of the polypeptides in a living body and to assist diagnosis and treatment, may easily and massively be obtained from the hybridoma.

The diagnostic product for autoimmune diseases of the present invention contains the polypeptide of the present invention, and examines antigen-antibody reaction against an antibody contained in serum of a person to be diagnosed. The diagnostic product may easily carry out the diagnosis for GVHD or GVL.

The diagnostic kit of autoimmune diseases of the present invention contains the polypeptide of the present invention, and screens serum of a GVHD or a GVL patient. The diagnostic kit may easily carry out the diagnosis for GVHD or GVL.

The examining kit of the present invention contains the antibody of the present invention, and may analyze living tissues of an AML, a CML, a GVHD, or a GVL patient. It is possible to easily analyze the localization of the polypeptides in a living body, and may assist diagnosis and treatment by using the examining kit.

The remedy for cancer of the present invention contains at least one selected from the polynucleotide and the polypeptide, and treat cancer by strengthening the defense mechanism of a living body against cancer cells. Therefore, the administration of the remedy for cancer of the present invention may positively induce GVL condition in the living body of the AML or the CML patient, and thus may effectively treat cancer. In addition, the remedy for cancer may be composed of a mixed remedy containing at least one of the polynucleotides and at least one of the polypeptides. This composition may further strengthen the defense mechanism of the living body.

The vaccine of the present invention contains at least one selected from the polynucleotides and the polypeptides, and makes a living body to acquire the defense mechanism of the living body against cancer cells to prevent the onset and increasing cancer cells. The vaccine may mainly be administered into a person who does not get AML or CML or a person who is likely to genetically get the AML or the CML to effectively prevent the person from getting the AML or the CML. In addition, the vaccine may be composed of a mixed vaccine containing at least one of the polynucleotides and at least one of the polypeptides. This composition may further increase the preventive effect.

The DNA chip of the present invention is a DNA chip containing the polynucleotide of the present invention. When the DNA chip is used, a monitoring of gene expression may easily be carried out.

### EXAMPLES

Examples that embody the above embodiments will now be described.

(Screening of cDNA Library)

Patient A (a woman of 42 year-old) of the high risk case, who is diagnosed with AML M1 Ph1+, was transplanted bone marrow obtained from her elder sister, who matches HLA, in her primary remission phase. The patient A retained the complete remission for a while. However, one and half years later, the reduction of myodynamia in her whole body with muscular atrophy appeared. For this symptom, the treatment with an immunosuppressant and the like was carried out. However, she finally died of the respiratory insufficiency due to the chronic GVHD.

Cell lysates of various cell lines were analyzed with Western blotting method using mixed serum in which the serum of patient A when the muscular atrophy appeared was mixed with the serum of CML patients B to D. As a result, it was proven that the mixed serum caused strongly specific antigen-antibody reaction in human leukemia cell K562 cells.

Next, cDNA library was produced from the K562 cells using a random primer (a hexamer). On about 10⁶ clones contained in this library, a phage λ, which contains the cDNA encoding the polypeptide that combines with the antibody in the mixed serum, was screened using serological analysis of antigens by recombinant cDNA expression cloning method (SEREX method). Ten kinds of clones that finally reacted strong-positively were isolated and identified.

(Characterization)

The obtained ten kinds of clones were determined their base sequences. As a result, eight kinds of base sequences represented by SEQ ID NO: 1 to SEQ ID No. 8 (i.e., pn1 to pn8) were determined. Here, remaining two kinds of clones overlap with pn3 and pn7, respectively. Here, the clone that overlapped with pn3 (reacting with the serum of patient B) reacted with the serum of patient C. These results and examining results of origin of the serum (patient) in which the gene product of each clone combined are shown in Table 1.

TABLE 1

| | Homology Search | Disease name or | |
|---|---|---|---|
| | Result | Symptom | Patient |
| pn1 | novel | AML, GVHD after BMT | A |
| pn2 | novel | AML, GVHD after BMT | A |
| pn3 | novel | CML | B, C |
| pn4 | novel | CML | C |
| pn5 | novel (kif1a) | CML | C |
| pn6 | LEDGF | CML | C |
| pn7 | Sip1 | CML | C |
| pn8 | HSP70 | CML | D |

(Cloning of pn9 and pn10)

An oligo-DNA primer that complementarily combines with both ends of each of pn1 and pn2, respectively, was artificially synthesized. The cDNA library was screened using both primers, while each of pn1 and pn2 was extended to the 5′ end side and the 3′ end side. Finally, a base sequence that is regarded as ORF including pn1 or pn2, respectively, i.e., base sequence of pn9 or pn10, was determined.

(Homology Search)

Homology search regarding the base sequences of pn1 to pn10 with respect to base sequences of well known nucleic acids that were already registered on the nucleic acid sequence databases was carried out using BLASTN(GENBANK). Regarding some clones, homology search regarding the amino acid sequences encoded by the base sequences with respect to amino acid sequences of well known polypeptides that were already registered on the amino acid sequence databases was carried out using BLASTP(Swiss Plot). In addition, regarding the amino acid sequences, at least one kind of searches selected from the search of the localization using RESORT and the search of the protein motif using MOTIF was carried out.

Useful information extracted from these search results was shown at the column of “Homology Search Result” of Table 1. It was predicted that pn3 and pn4 have encoded an amino acid sequence with zinc finger domain, and pn5 has encoded an amino acid sequence homologous with the kinesin. In addition, it was predicted that pn9 and pn10 have encoded a protein localized in the nucleus since these have high hydrophobic sites and a plurality of phosphorylation sites.

In the column of “Homology Search Result” shown in Table 1, “novel” refers that a base sequence and an amino acid sequence with specifically high homology were not confirmed, and pn5 has slightly high homologous with kinesin-like protein (kifla). In Table 1, “LEDGF” refers human epithelium lentis derived growth factor, “Sip1” refers human splicing factor Sip1, and “HSP70” refers 70kD (Dalton) heat shock protein.

(Verification of Reactivity for Normal Human Serum)

For the polypeptide produced by phage λ containing each polynucleotide of pn1 to pn10, antigen-antibody reaction was carried out using mixed serum that was obtained by mixing serum obtained from a plurality of healthy persons. The antigen-antibody reaction was not confirmed for each of clones.

At least one kind of polynucleotides selected from pn1 to pn10 may be used for producing a DNA chip. By using the DNA chip, a monitoring of gene expression for the polynucleotide of each of pn1 to pn10 may easily be carried out.

The polypeptide of the present invention may be used for a method of diagnosing autoimmune diseases characterized by using the polypeptide for screening serum of a GVHD or a GVL patient. By using the polypeptide, the diagnosis for GVHD or GVL may easily be carried out.

The antibody of the present invention may be used for a method of examining diseases characterized by analyzing tissues in a living body of an AML, a CML, a GVHD, or a GVL patient. The use of the antibody may easily analyze the localization of the polypeptide in a living body, and thus may assist diagnosis and treatment.

1. A polynucleotide comprising a base sequence that is identical or substantially identical with a base sequence represented by SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID No: 8, SEQ ID NO: 9, and SEQ ID NO: 10.

2. A recombinant vector containing the polynucleotide according to claim 1.

3. A transformant containing the recombinant vector according to claim 2.

4. The polynucleotide according to claim 1 labeled with radioisotope, enzyme, or fluorochrome.

5. A polypeptide comprising an amino acid sequence that is identical or substantially identical with an amino acid sequence encoded by the polynucleotide according to claim 1.

6. An antibody against the polypeptide according to claim 5.

7. A hybridoma producing the antibody according to claim 6.

8. A diagnostic product for autoimmune diseases containing the polypeptide according to claim 5.

9. A diagnostic kit of autoimmune diseases for screening serum of a GVHD or a GVL patient, wherein the diagnostic kit contains the polypeptide according to claim 5.

10. A kit for examining diseases for analyzing living tissues of an AML, a CML, a GVHD, or a GVL patient, wherein the kit contains the antibody according to claim 6.

11. A remedy for cancer for treating a cancer by strengthening the defense mechanism of a living body against cancer cells, wherein the remedy contains the polynucleotide according to claim 1.

12. A remedy for cancer for treating a cancer by strengthening the defense mechanism of a living body against cancer cells, wherein the remedy contains the polypeptide according to claim 5.

13. A remedy for cancer for treating a cancer by strengthening the defense mechanism of a living body against cancer cells, wherein the remedy contains the polynucleotide according to claim 1 and the polypeptide including an amino acid sequence that is identical or substantially identical with an amino acid sequence encoded by said polynucleotide.

14. The remedy for cancer according to any one of claims 11 to 13, wherein the remedy is prepared as an injection.

15. A vaccine for preventing development of a cancer by allowing a living body to acquire the defense mechanism of the living body against cancer cells, wherein the vaccine contains the polynucleotide according to claim 1.

16. A vaccine for preventing development of a cancer by allowing a living body to acquire the defense mechanism of the living body against cancer cells, wherein the vaccine contains the polypeptide according to claim 5.

17. A vaccine for preventing development of a cancer by allowing a living body to acquire the defense mechanism of the living body against cancer cells, wherein the vaccine contains the polynucleotide according to claim 1 and the polypeptide including an amino acid sequence that is identical or substantially identical with an amino acid sequence encoded by said polynucleotide.

18. The vaccine according to any one of claims 15 to 17, wherein the vaccine is prepared as an injection.

19. A DNA chip containing the polynucleotide according to claim 1.
